(19) United States
(12) Patent Application Publication
(10) Pub. No.: US 2005/0228055 A1
(43) Pub. Date: Oct. 13, 2005

(54) Substituted nitrobenzene derivatives as pharmaceutical and other uses athereof

(76) Inventors: Tewa Men, Beijing (CN); Yangyang Men, Beijing (CN)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/144,787

(22) Filed: Jun. 6, 2005

Related U.S. Application Data
(62) Division of application No. 10/129,367, filed on Jul. 24, 2002.

(30) Foreign Application Priority Data
Nov. 5, 1999 (CN) 99123684.X

(51) Int. Cl. A61K 31/135

(52) U.S. Cl. 514/741

(57) ABSTRACT

The present invention relates to a use of substituted nitrobenzene derivatives of general Formula I in medicine and health food, the pharmaceutical compositions comprising substituted nitrobenzene derivatives of general Formula I and the methods thereof for the prophylaxis and treatment of diseases.

### FIELD OF THE INVENTION

The present invention relates to substituted nitrobenzene derivatives of general Formula I as medicine and health food, a pharmaceutical composition comprising a substituted nitrobenzene derivative of general Formula I and the method thereof for the prophylaxis and treatment of diseases.

### BACKGROUND ART

Nippon Kagaku Zaeshi 80, 476-86 (1953) discloses some nitrobenzene compounds, but nothing is reported about any use thereof.

### OBJECTIVE OF THE INVENTION

The objective of the present invention is to develop a use of substituted nitrobenzene derivatives.

### SUMMARY OF THE INVENTION

After extensive and intensive researches, the inventor has unexpectedly found that substituted nitrobenzene derivatives of the following general formula I exhibit many excellent bioactivities on animal, e.g. immunoregulation, antisepsis, anti-virus, control of abnormal cell proliferation, cardio-cerebral vascular system, mechanical or physical injury, blood system, endocrine system, drug addiction, central nervous system, etc. The present invention is based on the above findings.

The first aspect of the present invention relates to substituted nitrobenzene derivatives of general Formula I useful for the treatment of animal diseases.

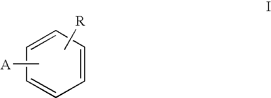

Wherein, A is one, two or three nitro groups, at 1, 3, or 5 position on the benzene ring; R is one, two or three linear or branched C₁₋₆ alkyl, at 2, 4 or 6 position on the benzene ring.

The second aspect of the present invention relates to a pharmaceutical composition for the treatment of animal diseases, comprising a therapeutically-effective amount of substituted nitrobenzene derivatives of general Formula I, pharmaceutical carrier and excipient,

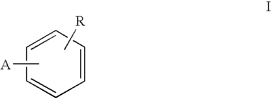

Wherein, A is one, two or three nitro groups, at 1, 3, or 5 position on the benzene ring; R is one, two or three linear or branched C₁₋₆ alkyl groups, at 2, 4 or 6 position on the benzene ring.

Another aspect of the present invention relates to a use of substituted nitrobenzene derivatives of general Formula I for the preparation of health food.

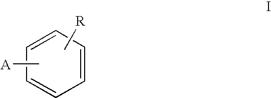

Wherein, A is one, two or three nitro groups, at 1, 3, or 5 position on the benzene ring; R is one, two or three linear or branched C₁₋₆ alkyl, at 2, 4 or 6 position on the benzene ring.

The another aspect of the present invention relates to a use of substituted nitrobenzene derivatives of general Formula I for the preparation of pharmaceuticals for the treatment of animal diseases.

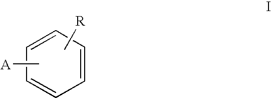

Wherein, A is one, two or three nitro groups, at 1, 3, or 5 position on the benzene ring; R is one, two or three linear or branched C₁₋₆ alkyl groups, which are the same or different, at 2, 4 or 6 position on the benzene ring.

Still another aspect of the present invention related to a use of a pharmaceutical composition for the preparation of pharmaceuticals for the treatment of animal diseases, wherein said composition comprises a therapeutically-effective amount of substituted nitrobenzene derivatives of general Formula I, pharmaceutical carrier and/or excipient.

The present invention also relates to a use of substituted nitrobenzene derivatives of general Formula I for the preparation of disinfectants,

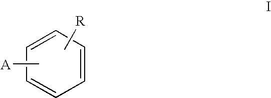

Wherein, A is one, two or three nitro groups, at 1, 3, or 5 position on the benzene ring; R is one, two or three linear or branched C₁₋₆ alkyl groups, wherein each R may be the same or different and at 2, 4 or 6 position on the benzene ring when R groups more than two are present.

The present invention also relates to a use of substituted nitrobenzene derivatives of general Formula I for the preparation of cosmetics,

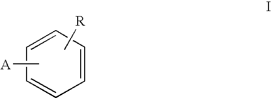

Wherein, A is one, two or three nitro groups, at 1, 3, or 5 position on the benzene ring; R is one, two or three linear or branched C₁₋₆ alkyl groups, which are the same or different, at 2, 4 or 6 position on the benzene ring.

Furthermore, the present invention relates to a method of treatment of animal diseases, comprising administrating the subject a therapeutically-effective amount of substituted nitrobenzene derivatives of general Formula I, or the pharmaceutical composition containing them.

### DETAILED DESCRIPTION OF THE INVENTION

The term “animals” used in the present description denotes mammal, especially human.

The term “linear or branched C₁₋₆ alkyl groups” used in the present description denotes linear or branched alkyl groups containing 1-6 carbon atoms, e.g. methyl, ethyl, propyl, isopropyl, n-butyl, isobutyl, sec-butyl, tert-butyl, amyl, neoamyl or hexyl.

The term “diseases or symptoms” used in the present description embraces diseases or symptoms caused by dysimmunity, bacteria, fungi, viruses, neurodegeneration, imbalance of the blood system, abnormal cell proliferation, endocrine dysfunction, abnormal changes of tissues, drug addiction and physical or mechanical factors, etc. Said diseases or symptoms include, e.g. benign tumors, malignant tumors, AIDS caused by HIV (Human Immunodeficiency Virus), virus hepatitis, allergic diseases, bronchitis, asthma, diabetes mellitus, uremia, nephritis, pulmonary tuberculosis, dysentery, poliomyelitis, chickenpox, herpes zoster, parotitis, cholera, influenza, scarlet fever, diphtheria, pertussis, typhoid, viral encephalitis, malaria, visceral leishmaniasis, brucellosis, rabies, plague, anthrax, leptospirosis, aplastic anemia, nutritional anemia, hemolytic anemia, Mediterranean anemia, hemophilia, dysfunction of blood coagulation, thrombocytopenic purpura, granulocytopenia, leukemia, myeloproliferative disease, arrhythmia, rheumatic heart disease, hyperlipemia, hypercholesterolemia, angina pectoris, hypertension, rheumatic valvular heart disease, myocarditis, endocarditis, pericarditis, cardiac neurosis, atherosclerosis, cerebrovascular spasm, cerebral apoplexy, cerebral thrombosis, peripheral vascular disease, rheumatoid arthritis, gastritis, gastric ulcer, enteritis, optic neuritis, conjunctivitis, otitis media, paranasal sinusitis, stomatitis, pharyngitis, gingivitis, periodontitis, tonsillitis, parotitis, cholecystitis, cystitis, pancreatitis, arteritis, phlebitis, vaginitis, vulvitis, orchitis, salpingitis, ovaritis, endometritis, tumor of cervix, prostatitis, soft tissue inflammation, osteomyelitis, myelitis, peritonitis, cecitis, psoriasis, dermatasclerosis, neurodegenerative diseases such as anxiety, depression, Huntington's disease, Parkinson's disease, Alzheimer's disease, etc, drug addiction to papaverine, morphine, heroin, cocaine, “ice” (methamphetamine hydrochloride), opium, dolantin, etc, viral dermatosis, coccigenic dermatosis, bacillary dermatosis, fungal dermatosis, spirochetal dermatosis, zoogenetic dermatosis, occupational dermatosis, neurotic dermatosis, allergodermia, physicogenic dermatosis, photogenous dermatosis, allergic dermatosis, lichen planus and lichenoid rash, erythemoid dermatosis, erythroderma desquamativum, vesicular dermatosis, keratinizing dermatosis, pigmentary dermatosis, dermovascular disease and lymphodermia, nutritional and metabolic dermatosis, disease of dermis and hypodermis, reticulosis, skin, syndrom, granuloma annulare, pustuloderma, dermatitis continuee of extremities, edeomycodermatitis such as cervical erosion, neurodermatitis, ulcerative disease of gastrointestinal tract and mouth cavity, burn, scald, cold injury, trauma, bone injury, soft tissue injury, snakebite, bee sting, etc.

The compounds of Formula I according to the present invention, wherein A is one nitro group at 1 position on the benzene ring, R is CH₃, C(CH)₃ and CH₃ at 2, 4 and 6 position respectively on the benzene ring, are preferred.

The compounds of Formula I according to the present invention, wherein A is preferably three nitro groups at 1, 3 and 5 position on the benzene ring, R is preferably CH₃, C(CH)₃ and CH₃ at 2, 4 and 6 position respectively on the benzene ring.

The compounds of Formula I according to the present invention, wherein A is preferably two nitro groups at 3 and 5 position on the benzene ring, R is preferably CH₃, C(CH)₃ and CH₃ at 2, 4 and 6 position respectively on the benzene ring.

The compounds of Formula I according to the present invention, wherein A is preferably one nitro group at 1 position on the benzene ring, R is preferably C(CH)₃, CH₃, and CH₃ at 2, 4 and 6 position respectively on the benzene ring.

The compounds of Formula I according to the present invention, are preferably selected from the following group:
1,3,5-trinitro-2,6-dimethyl-4-tert-butyl benzene;
1,3-dinitro-2,6-dimethyl-4-tert-butyl benzene;
1,5-dinitro-2,6-dimethyl-4-tert-butyl benzene;
3,5-dinitro-2,6-dimethyl-4-tert-butyl benzene;
1-nitro-2,6-dimethyl-4-tert-butyl benzene;
3-nitro-2,6-dimethyl-4-tert-butyl benzene;
5-nitro-2,6-dimethyl-4-tert-butyl benzene;
1,3,5-trinitro-2,4,6-tert-butyl benzene;
1,3-dinitro-2,4,6-tri-tert-butyl benzene;
3,5-dinitro-2,4,6-tri-tert-butyl benzene;
1,5-dinitro-2,4,6-tri-tert-butyl benzene;
1-nitro-2,4,6-tri- tert-butyl benzene;
3-nitro-2,4,6-tri-tert-butyl benzene;
5-nitro-2,4,6-tri-tert-butyl benzene;
1,3,5-trinitro-2,4,6-tri-methyl benzene;
1,3-dinitro-2,4,6-tri-methyl benzene;
3,5-dinitro-2,4,6-tri-methyl benzene;
1,5-dinitro-2,4,6-tri-methyl benzene;
1-nitro-2,4,6-tri-methyl benzene;
3-nitro-2,4,6-tri-methyl benzene;
5-nitro-2,4,6-tri-methyl benzene; and
1-nitro-2,6-dimethyl-4-tert-butyl benzene

According to the present invention, it is found that the compounds of Formula I may act selectively on the metabolic processes of important bio-active substances through the control of central nervous system-mediated body information network, to enhance the synthesis of physio-active substances which is responsible for the regulation of metabolism and bio-active substances which resist the endogenous and exogenous pathogenic factors, and to accelerate the cell division as well, resulting in active repair of injury and prompt improvement of the defense function against pathogenic factors, including barrier action, immune surveillance and recognition, phagocytosis and killing, detoxification and clearing, specific and non-specific immunity, while directly clearing the pathogenic factors. In detail, said compound of Formula I may exhibit beneficial bio-activities through the following ways:

1. Said compound of Formula I can rapidly improve specific and non-specific immunity, including humoral immunity, cellular immunity, phagocytosis of reticuloendothelial system and the activity of NK cell under both normal and abnormal conditions, and thus enhance the recognition, surveillance, killing, phagocytosis and clearance of such foreign bodies as bacteria, viruses and tumor cells. In particular, said compound of Formula I may, in sensitive animals, activate the cortex cells of thymus which belongs to central lymph organ and the precursor cells in bone marrow, promote cell division and proliferation of immunoblast, increase the number of lymphocyte in peripheral circulation, and promote the conversion of lymphocyte, the synthesis of antibodies by plasma cell, the phagocytosis of macrophage and phagocyte, while protect the stability of cell, avoid tissue lysis in the meantime, especially towards cytozoic pathogens such as viruses and Mycobacterium tuberculosis, tumor cells, and diseases caused by bacteria, fungi and viruses.

2. Said compound of Formula I can stabilize the lysosome membrane and decrease the release of hydrolase. Since hydrolase acts to decompose proteins, to cleave nucleic acid and mucopolysaccharide, leading to destruction of tissue cell, decomposition of connective tissue, and release of histamine, serotonin and kinin, which initiate a series of inflammatory reactions by angiectasis, increasing vascular permeability and aggregation of leucocytes, the stability of lysosome membrane may not only reduce the above mentioned inflammatory reactions, in particular, the exudation, edema and capillary hyperpermeability during the early phase of inflammation, tissue lysis by leucocytes and phagocytes, so as to alleviate such symptoms as redness, swelling, calor and tenderness, but also inhibit the hyperplasia of fibroblast during the late phase of inflammation to avoid granulation, adhesion and scarring. Also, it may improve or cure allergic diseased caused by endogenous histamine and produce antipruritic effect; inhibit the release of sensitizing substances by mastocyte to alleviate or eliminate the bronchospasm and myxedema, and thus produce antiasthmatic and antiussive effects. Due to the ability to improve stability of antibodies and lysosome membrane, said compound of Formula I may avoid and eliminate such allergic diseases as autoimmune diseases, anaphylaxis, and diseases due to connective tissue proliferation.

3. Said compounds of Formula I anticipate the tissue repair during the late phase of inflammation, in particular, enhance the regeneration and renew of tissue cells, such as smooth muscle cells, skeletal muscle cells, dermis and mucoca, inhibit fibroblast proliferation, to prevent the granulation and scarring, while leading to fast healing and delayed senility.

4. Said compounds of Formula I may act upon hemoglobin, platelet and granulocyte to enhance hematopoiesis e.g. synthesis of hemoglobin and cell division, and thus prevent and treat diseases of the blood system, alleviate fatigue, increase oxygen supply to heart, brain and other tissue cells, improve sleeping disorders, memory or intelligence.

5. Said compounds of Formula I may act selectively on the genetic center of enkephalin, through specific binding to the opium receptors, to achieve analgesia and abstinence.

6. Said compounds of Formula I may selectively participate the process of bio-synthesis and release of neurotransmitter, to coordinate the functions of cholinergic and adrenergic nerves, to regulate the functions of vegetative and motor nerves, e.g. visceral smooth muscle, gland effector, skeletal muscle, vessels and cardiac muscle, and thus help the body to rest and accumulate energy, to regulate the blood system, heart rate, digestive function, exocrine and endocrine of the body.

7. Said compounds of Formula I also participate the metabolism of bio-catalyst, i.e. various bio-enzymes e.g. synthase, decomposase, tool enzyme, steapsin, pancreatic amylase, trypsase, etc, and the secretion of insulin, and thus regulate the metabolism of lipid, carbohydrate and protein, to prevent and treat diseases or symptoms related to abnormal metabolism of lipid, carbohydrate and protein, e.g. diabetes mellitus, fatty liver, gout, atherosclerosis, etc, as well as detoxification in cases of ethylism, snakebite and bee sting.

Therefore, the present invention relates to compounds of Formula I preferably used for the prophylaxis and/or treatment of diseases, injury or symptoms cause by dysimmunity, bacteria, fungi, viruses, neurodegeneration, imbalance of the blood system and cardio-cerebral vascular system, abnormal cell proliferation, endocrine dysfunction, abnormal changes of antibodies and tissues, inflammation, drug addiction and physical or mechanical factors, etc, wherein A and R are defined as above.

More particularly, according to the present invention, compounds of Formula I used for the prophylaxis and treatment of AIDS (Acquired Immunodeficiency Syndrome), benign tumors, malignant tumors, virus hepatitis, tuberculosis, poliomyelitis, typhoid, bacillary dysentery, cholera, influenza, chickenpox, herpes zoster, parotitis, scarlet fever, diphtheria, pertussis, encephalitis B, viral encephalitis, malaria, visceral leishmaniasis, rabies, plague, anthrax, brucellosis, leptospirosis, and the use thereof for the preparation of pharmaceuticals for treatment of the above diseases or symptoms.

According to the present invention, compounds of Formula I used for the prophylaxis and treatment of arrhythmia, rheumatic heart disease, angina pectoris, coronary atherosclerosis, hyperlipemia, hypercholesterolemia, hypertension, myocarditis, endocarditis, pericarditis, cardiac neurosis, cerebrovascular spasm, cerebral thrombosis, peripheral vascular disease, and the use thereof for the preparation of pharmaceuticals for treatment of the above diseases or symptoms.

According to the present invention, compounds of Formula I used for the prophylaxis and treatment of aplastic anemia, nutritional anemia, hemolytic anemia, Mediterranean anemia, hemophilia, dysfunction of blood coagulation, thrombocytopenic purpura, granulocytopenia, leukemia, leukopenia due to chemotherapy and radiotherapy, and the use thereof for the preparation of pharmaceuticals for treatment of the above diseases or symptoms.

According to the present invention, compounds of Formula I used for the prophylaxis and treatment of diabetes mellitus, uremia, nephritis, nephrotic syndrome, and the use thereof for the preparation of pharmaceuticals for treatment of the above diseases or symptoms.

According to the present invention, compounds of Formula I used for the prophylaxis and treatment of anxiety, depression, Huntington's disease, Parkinson's disease, Alzheimer's disease (senile dementia) or presenile dementia, and the use thereof for the preparation of pharmaceuticals for treatment of the above diseases or symptoms.

According to the present invention, compounds of Formula I used for the prophylaxis and treatment of osteomyelitis, encephalitis, optic neuritis, conjunctivitis, otitis media, paranasal sinusitis, stomatitis, pharyngitis, gingivitis, periodontitis, tonsillitis, bronchitis, pneumonia, gastroenteritis, cholecystitis, cystitis, pancreatitis, arteritis, phlebitis, neuritis, vaginitis, vulvitis, orchitis, salpingitis, ovaritis, endometritis, prostatitis, psoriasis, and the use thereof for the preparation of pharmaceuticals for treatment of the above diseases or symptoms.

According to the present invention, compounds of Formula I used for the prophylaxis and treatment of systemic lupus erythematosus, polymyositis, dermatomyositis, dermatasclerosis, rheumatoid arthritis, rheumatic heart disease, rheumatic arthritis, ulcerative disease such as ulcerative colitis, pemphigus, allergic rhinitis, allergic dermatitis, allergic purpura, asthma, serum disease, allergic granulocytopenia, and the use thereof for the preparation of pharmaceuticals for treatment of the above diseases or symptoms.

According to the present invention, compounds of Formula I used for the prophylaxis and treatment of viral dermatosis, coccigenic dermatosis, bacillary dermatosis, fungal dermatosis, spirochetal dermatosis, zoogenetic dermatosis, occupational dermatosis, neurotic dermatosis, allergodermia, physicogenic dermatosis, photogenous dermatosis, allergic dermatosis, lichen planus and lichenoid rash, erythemoid dermatosis, erythroderma desquamativum, vesicular dermatosis, keratinizing dermatosis, pigmentary dermatosis, dermovascular disease and lymphodermia, nutritional and metabolic dermatosis, disease of dermis and hypodermis, reticulosis, skin syndrom, granuloma annulare, pustuloderma, dermatitis continuee of extremities, edeomycodermatitis such as cervical erosion, neurodermatitis, ulcerative disease of gastrointestinal tract and mouth cavity, and the use thereof for the preparation of pharmaceuticals for treatment of the above diseases or symptoms.

According to the present invention, compounds of Formula I used for abstinence from papaverine, morphine, heroin, cocaine, “ice” (methamphetamine hydrochloride), opium, dolantin, and the use thereof for the preparation of pharmaceuticals for abstinence from the above drug addiction.

According to the present invention, compounds of Formula I used for the treatment of burn, scald, cold injury, trauma, bone injury, soft tissue injury, snakebite, bee sting, and the use thereof for the preparation of pharmaceuticals for the treatment of the above injuries.

The present invention also relates to pharmaceutical compositions comprising at least one compound of Formula I as the active ingredient, pharmaceutical carrier or excipient. The present pharmaceutical compositions usually contain 0.1-90% by weight compounds of Formula I. According to the present invention, said pharmaceutical compositions may be prepared by known methods in the art. Said compounds of Formula I may be prepared in combination with one or several solid or liquid pharmaceutical carriers and/or excipients, in suitable administration route and dosage form for human and veterinary application as needed.

According to the present invention, said compounds of Formula I or the pharmaceutical compositions comprising them, may be administered, in unit dosage form, intestinally, parenterally or topically, e.g. orally, intramuscularly, subcutaneously, intranasally, by oral absorption, intravenously, percutaneously, intraperitoneally, rectally, etc. Administrative dosage are as follows: tablet, capsule, drop, aerosol, pill, powder, liquor, suspension, emulsion, granule, ointment, suppository, freeze-dried injection, etc, which could be regular preparation, delayed-released preparation, controlled-released preparation and various micro-granule delivery system. In case of tablet, various carriers known in the art may be used, e.g. dilutent and resorbent such as starch, dextrin, calcium sulfate, kaolin, microcrystalline cellulose, aluminium silicate, etc; wetting agent and adhesives such as water, glycerin, polyethylene glycol, ethanol, propanol, starch mucilage, dextrin, syrup, honey, glucose solution, acacia, gelatin, carboxymethylcellulose sodium, shellac, methylcellulose, potassium phosphate, polyvinylpyrrolidone, etc; disintegrating agent, such as dried starch, alginate, agar powder, laminaran, sodium bicarbonate and citric acid, calcium carbonate, polyoxyethylene sorbitol aliphatic ester, lauryl sodium sulfate, methylcellulose, ethylcellulose, etc; disintegration inhibiting agent, such as sucrose, tristearin, cacao butter, hydrogenated oil, etc; absorption accelerator, such as quaternary ammonium salt, lauryl sodium sulfate, etc; lubricant, such as talc, silica, corn starch, stearate, boric acid, fluid wax, polyethylene, etc. The tablet may be further formulated into coated tablet, e.g. sugar-coated tablet, film-coated tablet, enteric-coated tablet, or double-layer tablet and multi-layer tablet. In the case of pill, various carriers known in the art may be used, e.g. dilutent and resorbent, such as glucose, lactose, starch, cacao butter, hydrogenated vegetable oil, polyvinylpyrrolidone, kaolin, talc, etc; adhesives, such as acacia, bassora gum, gelatin, ethanol, honey, liquid sugar, rice paste or flour paste, etc; disintegrating agent, such as agar powder, dried starch, alginate, lauryl sodium sulfate, methylcellulose, ethylcellulose. In case of suppository, various carriers known in the art may be used, e.g. polyethylene, lecithin, cacao butter, higher alcohols, esters of higher alcohols, gelatin, semi-synthetic glyceride, etc. In the case of capsule, it may be prepared by mixing said compounds of Formula I as active ingredient with the above mentioned carriers, followed by placing the mixture into a hard gelatin capsule or soft capsule. Also, said compounds of Formula I may be applied in the following dosage forms: microcapsules, suspension in an aqueous phase, hard capsule, or injection. In the case of injection, such as liquor, emulsion, freeze-dried injection, and suspension, all the dilutents common in the art may be used, e.g. water, ethanol, polyethylene glycol, propylene glycol, oxyethylated isostearyl alcohol, polyoxidated isostearyl alcohol, polyoxyethylene sorbitol aliphatic ester, etc. In addition, in order to obtain isotonic injection, a suitable amount of sodium chloride, glucose or glycerin may be added into the preparation, as well as regular cosolvent, buffer, pH adjusting agent, etc.

In addition, coloring agent, antiseptic, perfume, correctives, food sweetening agent or other materials may be added to the pharmaceutical preparation if necessary.

According to the present invention, the dosage of said compounds of Formula I depends on a variety of factors, including the nature and severity of the diseases, the sex, age, weight and individual reaction of the subject, the particular compound employed, the route and frequency of administration, etc. Said compounds of Formula I or the pharmaceutical compositions comprising them may be administered in single or divided dosage form, e.g. one to four doses per day.

The following examples and biological activity assays or clinical trials are presented for further illustrative purposes only and are not intended as a restriction on the scope of the invention.

I. Toxicologic Study

1. Acute Toxicity Test
(1) Oral Administration

Mice, weight: 20 g±2 g, male and female half by half. Nitrobenzene compounds were prepared as an aqueous suspension with a concentration of 20% and given to the fast mice three times per day by gastrogavage. 0.3 ml per time to a total of 18 g/kg, corresponding 600 times of the daily dosage for human. No death or lethal adverse reaction was observed for successive 7 days.
(2) Injection Administration

Mice, weight: 20 g±2 g, male and female half by half. Nitrobenzene compounds were prepared as an injection with a concentration of 2% and administered 0.4 ml each. Results: LD₅₀(ip)=18.07±4.52 g/kg, LD₅₀(iv)=14.19±2.11 g/kg.

2. Long-Term Toxicity Test

170 Wistar rats, weight: 86±8 g, male and female half by half. Gastrogavage. Nitrobenzene compounds were prepared as an aqueous suspension. 0.6, 2.0, 6 g/kg respectively (20, 70, 200 times the dosage for human). Continuous daily administration for 6 months. The mean gain in weight and the bioavailability rate of 3 experimental groups is higher, as compared with the control group. No significant difference was observed on peripheral hemogram, hepatic and renal function, and biochemical criteria such as blood sugar. No marked pathologic improvement was present in important organs. The results indicate that no lethal adverse reaction occur at the dosage of 6 g/kg/d.

3. Genetic toxicity test, traditional teratogenicity test, 18-month-diet carcinogenic test. The results show that nitrobenzene compounds demonstrate no noticeable teratogenic, mutagenic and carcinogenic actions.

II. Pharmacodynamics Study

1. Observations on analgesic, hemostatic, antipruritic, antiussive, antiasthmatic actions of the prepared pharmaceuticals on mice and guinea pigs.
(1) Body twisting. The reaction to intraperitoneal injection of glacial acetic acid after transgastric administration in mice was observed, and the twisting frequency of mice in 15 minutes was recorded. The twisting times were significantly reduced in experimental groups receiving three different dosages of said compounds (0.01 g/kg, 0.02 g/kg, 0.04 g/kg), as compared with the control group (P<0.01). The result indicates that said compounds demonstrate marked analgesic actions.
(2) Paw licking. The influence of local administration of said compounds on the threshold stimulus of histamine phosphate to surface of wound was observed and recorded. The threshold value, i.e. the total consumption of histamine phosphate (μg) was significantly higher in the experimental groups treated with three concentrations of said compounds (1%, 2%, 4%), than in the control group (P<0.01). Said compounds demonstrate marked antipruritic actions.
(3) Coughing induced by oxyammonia spray. The latency of cough following transgastric administration of said compounds and the coughing times in 3 minutes were observed and recorded. Said compounds significantly prolonged the latency and reduced coughing times in groups treated with three dosages (0.01 g/kg, 0.02 g/kg, 0.04 g/kg), as compared with the control group (P<0.01). The result indicates that said compounds demonstrate marked antiussive actions.
(4) Asthma induced by histamine spray. The duration from the start of spray to the happening of convulsion and turning of mice, i.e. the latency, after transgastric administration of said compounds, was observed and recorded. Said compounds significantly prolonged the latency in groups treated with three dosages (0.02 g/kg, 0.04 g/kg, 0.08 g/kg), as compared with the control group (P<0.01). The result indicates that said compounds demonstrate marked antiasthmatic actions.
(5) Bleeding of severed tail. After 3-day transgastric administration of said compounds, tails of the mice were cut at the level of 0.5 cm from the tips, and the bleeding time was clocked. Every 15 seconds, filter paper was used to absorb the blood drop, until the bleeding stopped naturally. The bleeding time was calculated. The result shows that said compounds significantly shorten the bleeding time in groups treated with three dosages (0.01 g/kg, 0.02 g/kg, 0.04 g/kg), as compared with the control group (P<0.01), indicating that said compounds demonstrate marked hemostatic actions.

2. Observations on prophylactic and therapeutic actions of said compounds on rheumatoid arthritis.
(1) Inhibition of adjuvant arthritis in rats.
(2) Inhibition of other experimental arthroncus.
(3) Influence on abdominal capillary permeability in mice.
(4) Influence on cotton ball granulation in rats.

The testing results prove that transgastric administration with three dosages (0.02 g/kg, 0.04 g/kg, 0.08 g/kg) demonstrates significant inhibition of adjuvant arthritis; prophylactic administration leads to significant inhibition of early inflammatory reaction and secondary swelling and significant inhibition of paw swelling of the other hind limb due to delayed hypersensitivity; therapeutic administration leads to significant repercussion of the adjuvant-treated limb, significant decreased secondary swelling, significant inhibition of paw swelling of the other hind limb due to delayed hypersensitivity, and significant abatement of such injuries as ear erythema and tail nodule in rats, as compared with the control group. The treated groups showed normal appetite, without significant difference in weight before and after dissection, or influence on the weight of thymus, spleen and adrenal gland. For inhibition test of other experimental arthroncus, significant inhibition was observed in case of paw swelling induced by carrageenan and formaldehyde in rats, and the cotton ball granulation was significantly inhibited as well. The capillary permeability was significantly reduced leading to decreased exudation. The above results indicate that said compounds demonstrate marked therapeutic and prophylactic actions on such allergic diseases as rheumatoid arthritis.

3. Observations on influence of said compounds on immunologic functions.
(1) Activity assay of NK-natural killer cells. Said compounds were administered in mice per os for 30 days. Said compounds showed significant enhancement on the activity of NK cell in three treated groups (0.004 g/kg, 0.04 g/kg, 0.12 g/kg), as compared with the control group (P<0.01).
(2) Serum agglutinin test was carried out in normal mice to observe the influence of said compounds on humoral immunity. The results indicate that said compounds significantly increased the serum anti-sheep RBC agglutinin value in three treated groups (0.05 g/kg, 0.10 g/kg, 0.20 g/kg), as compared with the control group (P<0.01) and thus demonstrated significant enhancement of humoral immunity in normal animals.
(3) The test of influence on serum agglutinin value in mice with tumors is carried out to observe the influence of said compounds on humoral immunity of S-180 mice with tumors. The results indicate that said compound significantly increase the serum anti-sheep RBC agglutinin value in three treated groups, as compared with the control group (P<0.01) and thus demonstrated significant enhancement of humoral immunity in S-180 mice with tumors.
(4) The test of influence on DTH in mice was carried out to observe the influence of said compounds on cellular immunity in mice. The results indicate that said compounds significantly increased the D-value of toe swelling in mice, as compared with the control group (P<0.01), and thus demonstrate significant enhancement of DTH in mice and cellular immunity of the body.
(5) The test of influence on clearance of carbon granules in mice was carried out to observe the influence of said compounds on non-specific immunity in mice. The results indicate that the clearance of three treated groups was significantly higher than the control group (P<0.01), as well as a higher phagocytic index α and said compounds demonstrate significant enhancement of phagocytic functions of reticuloendothelial system in normal mice.

4. Observations on inhibition of tumor growth.

Three groups receiving different dosages were set (0.02 g/kg, 0.08 g/kg, 0.16 g/kg), and compared with the control group.
(1) Significant inhibition of cervical carcinoma (U₁₄) in mice. The mean rate of tumor inhibition was 30.20%, 38.07% and 46.06% respectively. At the end of the test, the mortality was less than 20%, and the weight excluding tumor was not less than before the test. The test was repeated 3 times, and all yielded the same result.
(2) Significant inhibition of hepatic carcinoma (H₂) in mice. The mean rate of tumor inhibition was 29.53%, 38.20% and 44.6% respectively. At the end of the test, the mortality was less than 20%, and the weight excluding tumor was not less than before the test. The test was repeated 3 times, and all yielded the same result.
(3) Significant inhibition of carcinoma of stomach (Fc) in mice. The mean rate of tumor inhibition was 30.24%, 38.46% and 44.05% respectively. At the end of the test, the mortality was less than 20%, and the weight excluding tumor was not less than before the test. The test was repeated 3 times, and all yielded the same result.

The above results indicate that said compounds demonstrate significant inhibition of transplantation tumor growth in mice.

5. Observations on anti-fatigue and hypoxia tolerance actions of said compounds.

Three groups receiving different dosages were set (0.01 g/kg, 0.02 g/kg, 0.04 g/kg), using transgastric administration.
(1) The swimming duration of mice carrying 1/10 body weight in each group was observed and recorded. The results indicate that said compounds demonstrate significant anti-fatigue actions, as compared with the control group (P<0.01).
(2) The hypoxia tolerance time in mice was recorded (S, X±SD). The results show that said compounds demonstrate significant enhance of hypoxia tolerance in three treated groups, as compared with the control group (P<0.01).

6. Routine pharmacodynamics testing methods were adopted as follows: continuous transgastric administration for 20 days, 0.02 g/kg per day, bid. 24 hours after the last administration, blood samples were obtained to determine Hb, WBC and RBC. The results indicate that said compounds significantly increase peripheral WBC, RBC and Hb counting in mice, as compared with the control group (P<0.01).

7. Observations on the influence of said compounds on experimental hepatic injury in rabbits and acute hepatic injury in rats, the anxo-action on liver cell regeneration in experimental cirrhosis in rats, and the protection of excretory function of BSP and against TAA intoxication in mice.

The study shows that said compounds demonstrate significant inhibition of hepatic injury in rabbits, significant reduction of SGPT and significant protection for cells; inhibition of fatty degeneration, loosening and acidophilic degeneration of liver; inhibition of necrosis of liver cells. Said compounds significantly reduce SGPT and serum bilirubin, significantly alleviate such pathologic changes as cloudy swelling, fatty degeneration and pyrenolysis of liver cells in case of acute hepatic injury in rats. Said compounds demonstrate significant inhibition of SGPT elevation and pathologic changes in case of TAA induced acute hepatic intoxication in mice. Also, said compounds significantly reduce the content of hydroxyproline and total hepatic lipid, alleviate pathologic changes, enhance cell proliferation and the excretory function of liver, and reduce the retention volume of BSP in liver in mice.

8. Observations on the influence on ulcerative diseases.

The results show that said compounds at the dosage of 0.02 g/kg demonstrate significant inhibition of stress ulcer, ligature ulcer, cautery ulcer, ulcer induced by reserpine, and significant repair of mucosal lesions.

9. Observations on the influence on incomplete cerebral ischemia.

Bilateral carotid arteries were ligated in rats. Said compounds significantly not only decrease cerebrovascular permeability to prevent cerebral edema and pathologic changes of the brain, but also improve the circulation of cerebral pia mater, and, as such, help the brain recover from ischemia.

10. Observations on the antiviral and antibacterial actions.

The results show that in case of 3 and 30TCTD₅₀ virus infection, said compounds at the dosage of 0.01 g/kg demonstrate significant inhibition of cytopathy caused by influenza virus FM strain, enterovirus E (HO₁₁) and herpes virus type I, and of virus replication in vivo in mice. The results of antibacterial test show that said compounds demonstrate significant inhibition of the following 8 bacteria tested: Haemophilus influenzae, Streptococcus pneumoniae, Staphylococcus aureus, Escherichia coli, Pseudomonas aeruginosa, Mycobacterium tuberculosis, Shigella flexneri, yeast.

11. Clinical Examples
(1) Observations on symptoms and physical signs of 68 cases of late malignant tumor with pathologic diagnosis, who refused to receive any treatment except supporting treatment until death. Laboratory findings and rate of tumor inhibition were compared before and after the administration.

Among the 68 patients, there are 8 cases of carcinoma of large intestine, 8 cases of lung cancer, 11 cases of breast cancer, 9 cases of carcinoma of stomach, 12 cases of carcinoma of esophagus, 3 cases of acute lymphatic leukemia, and 1 case of melanoma.

0.01 g/kg/d, tid, oral administration for successive 60 days.

Results: Except 1 death 13 days after administration, 67 patients experienced significant improvement of cachexia. Among the 67 patients, 42 cases suffered from pain. 39 patients experienced significant improvement, 30 of which had been on dependent analgesic, while after the administration, they stopped the analgesic without marked abstinence symptoms. 14 cases suffered from hemoptysis and hematochezia, all of which experienced complete improvement. 3 cases suffered from jaundice, and all got complete remission. 9 cases suffered from transaminase elevation (above normal), 8 of which experienced complete recovery. 4 cases suffered from tuberculosis, and all were cured. 11 cases suffered from dysphagia, 10 of which experienced significant remission. 68 cases suffered from low hemoglobin, WBC and platelet counting, 65 of which got significant improvement. According to volume change of tumor, the total remission rate reaches 60%. The above data indicate that said compounds significantly improve life quality, alleviate symptoms, and exhibit effective analgesic, hemostatic, antipruritic, antiussive actions in the meantime of inhibition of tumor growth. Said compounds also demonstrate therapeutic actions on pathologic changes of liver cells with transaminase elevation, therapeutic actions on tuberculosis, effective protection of hemotopoiesis, as well as sharpen appetite, alleviate anxiety and improve sleep.
(2) Clinical observations on other diseases and symptoms.
(1) 8 cases of asthma, 11 cases of rheumatoid arthritis, 3 cases of nephritic syndrome, experienced significant remission and control. 24 cases of allergic rhinitis, 30 cases of paranasal sinusitis, 14 cases of systemic lupus erythematosus, 8 cases of pemphigus, 16 cases of allergic purpura, 14 cases of ulcerative colitis, 48 cases of stomatocace were completely cured.
(2) 4 cases of rheumatic valvular heart disease, 12 case of arrhythmia, 23 cases of hypertension, 36 cases of cardiac neurosis got significant control, 2 cases of bacterial endocarditis, 12 cases of pericarditis, 5 cases of myocarditis were completely cured.
(3) 43 cases of dermatosis, e.g. viral dermatosis, coccigenic dermatosis, bacillary dermatosis, fungal dermatosis, spirochetal dermatosis, zoogenetic dermatosis, occupational dermatosis, neurotic dermatosis, allergodermia, physicogenic dermatosis, photogenous dermatosis, allergic dermatosis, lichen planus and lichenoid rash, erythemoid dermatosis, erythroderma desquamativum, vesicular dermatosis, keratinizing dermatosis, pigmentary dermatosis, dermovascular disease and lymphodermia, nutritional and metabolic dermatosis, disease of dermis and hypodermis, reticulosis, skin syndrom, granuloma annulare, pustuloderma, dermatitis continuee of extremities, edeomycodermatitis such as cervical erosion, neurodermatitis, ulcerative disease of gastrointestinal tract and mouth cavity, were completely cured.
(4) 17 cases of chronic hepatitis, 45 cases of acute hepatitis, 54 cases of tuberculosis, 12 cases of poliomyelitis, 43 cases of dysentery, 18 cases of chickenpox, 16 cases of mumps, 22 cases of scarlet fever, 26 cases of encephalitis B, 3 cases of visceral leishmaniasis, 12 cases of brucellosis, 2 cases of AIDS, experienced significant curative effect and were completely cured.
(5) 3 cases of Mediterranean anemia, 8 cases of aplastic anemia, 5 cases of nutritional anemia, 13 cases of thrombocytopenic purpura, were completely cured. 9 cases of acute lymphatic leukemia got significant remission.
(6) 11 cases of myasthenia gravis, 72 cases of nervous headache, 12 cases of myelitis, 30 cases of intercostals neuritis, were completely cured.
(7) 136 cases of infection caused by trauma, burn, scald and other factors experienced quick healing without any scar left.
(8) As health food, anti-fatigue, anti-anemia, anti-decay actions, improvement of memory, promotion of growth and development, regulation of blood lipid, blood sugar, blood pressure and improvement of sexual function were observed in 129 cases.

10. As disinfectant, significant action was observed at the concentration of 0.001%.

11. In large-scale trials, said compounds exhibit significant therapeutic effects on controlling epidemic diseases in veterinary practice, such as rabbit fever, fowl plague, etc.

12. As nutrient, said compounds are effective to the growth of plant, human and domestic animals.

Synthetic Methods of Nitrobenzene Compounds

1. m-xylene and tert-butyl alcohol in proportion of 1:3 were placed in the reactor. A suitable amount of aluminum trichloride was employ as catalyst. With cooling by circulating ice water, it was stirred and mixed well and react for over 2 hours. The reaction solution was poured slowly into concentrated hydrochloride, in proportion of 3:1. When the solution became clear and transparent, the reaction was completed. The resulting solution was subjected to extraction with chloroform/water, intensively mixed, stand, and the organic phase was added to sodium bicarbonate. The process was repeated until the pH reached 6-8. The residual was distilled to afford 2,6-dimethyl-4-tert-butyl benzene.

2. To 2,6-dimethyl-4-tert-butyl benzene solution in the reactor, an equal amount of acetic anhydride was added. After intensive stirring and mixing, nitric acid was added slowly under circulating cooling. The reaction sample was identified by T.L.C to determine the completeness of the reaction.

3. The reaction solution was extracted with chloroform. The organic phase was dried over sodium carbonate followed by filtration. Allow standing, white acicular crystals precipitated to afford the product 1-nitro-2,6-dimethyl-4-tert-butyl benzene. Molecular weight: 207.273, formula: C₁₂H₁₇NO₂.

All compounds are white or creamy crystals, soluble in organic solvent, but not in water, with a scent of volatile oil, mild in taste. Mp: 80-82° C. Other nitrobenzene compounds belong to known technology, and are no more repeated here.

### EXAMPLE 1

### Suspension

100 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed and placed in a container and to it 100 ml glycerin was added. The mixture was heated to achieve dissolution and intensively mixed. Distilled water was added up to 1000 ml with intensive mixing to afford an emulsion with a concentration of 0.1%. It was then sealed and bottled, sterilized to give products. For external use or oral preparation.

### EXAMPLE 2

### Emulsion

150 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed and placed in a container and to it 100 ml sesame oil was added. The mixture was heated to achieve dissolution and intensively mixed. Distilled water was added up to 1000 ml with intensive mixing to afford an emulsion with a concentration of 0.15%. It was then sealed and bottled, sterilized to give products. For external use or oral preparation.

### EXAMPLE 3

### Aqueous for External Use

200 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed and placed in a container and to it 100 ml ethanol and a suitable amount of Tween were added. The mixture was heated to achieve dissolution and intensively mixed. Distilled water was added up to 1000 ml with intensive mixing to afford an aqueous solution with a concentration of 0.2%. It was then sealed and bottled, sterilized to give products for external use.

### EXAMPLE 4

### Capsule

300 g 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene, 300 g black soya bean powder, 140 g glucose, 60 g albumin, 200 g starch were weighed and subjected to grinding, mesh screening and intensive mixing, followed by capsule filling. Each capsule contained 0.2-0.33 g content, for oral pharmaceuticals and health food.

### EXAMPLE 5

### Injection

200 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl naphthalene(sodium salt) was accurately weighed and placed in a sterile container and to it 1000 ml water for injection was added. The mixture was heated to achieve dissolution and packed in 2 ml ampoules with a concentration of 4 mg/2 ml per ampoule. It was then sealed and sterilized to give products.

### EXAMPLE 6

### Disinfectant

100 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed and placed in a container and to it 100 ml a suitable amount of ethanol and Tween were added. The mixture was heated to achieve dissolution and intensively mixed. Distilled water was added up to 1000 ml with intensive mixing to afford a disinfectant a concentration of 0.01%.

### EXAMPLE 7

### Aqueous for External Use

135 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed and placed in a container and to it ethanol was added up to 800 ml followed by filtration. Still more ethanol was added through the filter to achieve 1000 ml. The mixture was stirred well to give the preparation. It was then sealed and refrigerated to be stored.

### EXAMPLE 8

### Shampoo

200 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed and placed in a container and to it 30 g glycerin, 50 ml ethanol were added and shaken well. Distilled water was added up to 2000 ml with intensive mixing to afford shampoo with a concentration of 0.1%. It was applied in baldess, seborrheic dermatitis of scalp and for antipruritic use.

### EXAMPLE 9

### Oral Liquid

250 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed and placed in a container and to it 100 ml sesame oil was added. After the mixture was heated to achieve dissolution, 200 ml honey was added and stirred well. Distilled water was added up to 1000 ml with intensive mixing to afford oral liquid with a concentration of 0.25%.

### EXAMPLE 10

### Suppository

100 mg 1-nitro-2,6-dimethyl-4-tert-butyl benzene or 3,5-dinitro-2,6-dimethyl-4- tert-butyl benzene or 1,3,5-trinitro-2,6-dimethyl-4-tert-butyl benzene was accurately weighed and to it melting semi-synthetic fatty glyceride was added fractionally, followed by quick stirring to the point of coagulation. It was then filled into suppository model, coagulated, scraped and then collected for packing.

### EXAMPLE 11

### Skin Emulsion

100 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed followed by grinding and mesh screening, and then placed in a mortar. Peanut oil was added and mixed well. Solution of calcium hydroxide was added slowly and ground to give a paste. More solution of calcium hydroxide was added up to 1000 ml to afford a emulsion for external application.

### EXAMPLE 12

### Ointment

100 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed followed by grinding and mesh screening, and then placed in a mortar. Glycerin was added and ground to achieve dissolution. Vaselin was added up to 1000 g, and ground well to afford ointment for external application or skin protection.

### EXAMPLE 13

### Powder

100 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed. Glucose was ground and mixed well followed by mesh screening to afford powder.

### EXAMPLE 14

### Powder

100 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed and to it 200 mg talc was added. Then boric acid and powder of zinc oxide, which were not ground in advance, were added in turn. A suitable amount of talc was added up to 200 mg and mixed well to afford powder.

### EXAMPLE 15

### Eye Ointment

200 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed and placed in a sterile mortar. A small amount of sterile liquid wax was added and ground to give fine paste. Then the ground substance was added fractionally, accompanied by grinding, to achieve 1000 g. It was then packed with aseptic manipulation, to afford eye ointment.

### EXAMPLE 16

### Eye Drops

50 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene sodium was accurately weighed and placed in a sterile container, to which 100 ml water for injection was added. The mixture was heated to achieve dissolution. Water for injection was added up to 500 ml. It was then bottled, flow-steam sterilized for 30 minutes to afford eye drops.

### EXAMPLE 17

### Ear Drops

200 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed and to it 400 ml ethanol was added. The mixture was heated to achieve dissolution followed by filtration. 300 ml glycerin was added and stirred well. Distilled water was added up to 500 ml. It was then packed to afford ear drops.

### EXAMPLE 18

### Nose Drops

200 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was accurately weighed and to it 40 ml ethanol was added. The mixture was heated to achieve dissolution. A suitable amount of Tween 80 was added and stirred well. Distilled water was added up to 5000 ml and mixed well to afford nose drops.

### EXAMPLE 19

### Tablet

Prepared according to the method known in the art. 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene and tablet, wherein said tablet comprised 1%-2% said compounds according to effective demand. The content of said compounds may be increased or decreased as well. According to known technology and equipment, 300 g compounds identified in the above examples, 200 g microcrystalline cellulose, 300 g anhydrous lactose, 200 g magnesium stearate, were formulated to afford tablet.

### EXAMPLE 20

### Oral Liquid

500 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was weighed and to it 300 ml sesame oil was added. The mixture was heated to achieve dissolution for later use. 100 g chrysanthemum, 100 g Japanese polygala, were weighed and steamed to give 500 ml liquid. After filtration, the liquid was poured to the previous solution, mixed well, bottled and steam sterilized to afford oral liquid.

### EXAMPLE 21

### Oral Nutrient

300 mg 1-nitro-2,6-dimethyl-4-tert-butyl benzene or 3,5-dinitro-2,6-dimethyl-4-tert-butyl benzene or 1,3,5-trinitro-2,6-dimethyl-4-tert-butyl benzene was weighed and to it 300 ml sesame oil was added. The mixture was heated to achieve dissolution. Honey at a dilution of 1:10 was added up to 5000 ml. It was then bottled and autoclaved.

### EXAMPLE 22

### Injection

A method for the preparation of nitrobenzene sodium injection.

500 ml water for injection was mixed with 500 mg powder of nitrobenzene sodium, followed by bottling and steam sterilization, to afford injection in 0.2 mg/2 ml and 0.3 mg/3 ml standards, for intramuscular and intravenous administration.

1-18. (canceled)

19. A method for the prophylaxis and treatment of diseases, injuries or symptoms caused by dysimmunity, bacteria, fungi, viruses, neurodegeneration, imbalance of the cardio-cerebral vascular system, endocrine dysfunction, abnormal changes of antibodies, inflammation, or physical or mechanical factors of drug addiction, comprising administrating an effective prophylaxis or treatment amount of at least one compound of Formula I to a patient who is suffering from at least one of said diseases, injuries or symptoms, 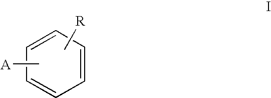
wherein, A is one, two or three nitro groups, at 1, 3, or 5 position on the benzene ring; R is one, two or three linear or branched C₁₋₆ alkyl groups, at 2, 4 or 6 position on the benzene ring.

20. The method according to claim 19, wherein, in the compound of Formula I, A is only one nitro group at 1 position on the benzene ring, R is CH₃, C(CH₃)₃ and CH₃ at 2, 4 and 6 position, respectively, on the benzene ring.

21. The method according to claim 19, wherein, in the compound of Formula I, A is three nitro groups at 1,3,5 positions, respectively, on the benzene ring, R is CH₃, C(CH₃)₃ and CH₃ at 2, 4 and 6 position, respectively, on the benzene ring.

22. The method according to claim 19, wherein, in the compound of Formula I, A is two nitro groups at 3,5 positions, respectively, on the benzene ring, R is CH₃, C(CH₃)₃ and CH₃ at 2, 4 and 6 position, respectively, on the benzene ring.

23. The method according to claim 19, wherein, in the compound of Formula I, A is one nitro group at 1 position on the benzene ring, R is C(CH₃)₃, CH₃, and CH₃ at 2, 4 and 6 position, respectively, on the benzene ring.

24. The method according to claim 19, wherein said compound of Formula I is selected from the group consisting of:
1,3,5-trinitro-2,6-dimethyl-4-tert-butyl benzene;
1,3-dinitro-2,6-dimethyl-4-tert-butyl benzene;
1,5-dinitro-2,6-dimethyl-4-tert-butyl benzene;
3,5-dinitro-2,6-dimethyl-4-tert-butyl benzene;
1-nitro-2,6-dimethyl-4-tert-butyl benzene;
3-nitro-2,6-dimethyl-4-tert-butyl benzene;
5-nitro-2,6-dimethyl-4-tert-butyl benzene;
1,3,5-trinitro-2,4,6-tri-tert-butyl benzene;
1,3-dinitro-2,4,6-tri-tert-butyl benzene;
3,5-dinitro-2,4,6-tri-tert-butyl benzene;
1,5-dinitro-2,4,6-tri-tert-butyl benzene;
1-nitro-2,4,6-tri-tert-butyl benzene;
3-nitro-2,4,6-tri-tert-butyl benzene;
5-nitro-2,4,6-tri-tert-butyl benzene;
1,3,5-trinitro-2,4,6-tri-methyl benzene
1,3-dinitro-2,4,6-tri-methyl benzene;
3,5-dinitro-2,4,6-tri-methyl benzene;
1,5-dinitro-2,4,6-tri-methyl benzene;
1-nitro-2,4,6-tri-methyl benzene;
3-nitro-2,4,6-tri-methyl benzene;
5-nitro-2,4,6-tri-methyl benzene; and
1-nitro-2,6-dimethyl-4-tert-butyl benzene.

25. The method according to claim 19, wherein said diseases or symptoms are: AIDS (Acquired Immunodeficiency Syndrome), virus hepatitis, tuberculosis, poliomyelitis, typhoid, bacillary dysentery, cholera, influenza, chickenpox, herpes zoster, parotitis, scarlet fever, diphtheria, pertussis, encephalitis B, viral encephalitis, malaria, visceral leishmaniasis, rabies, plague, anthrax, brucellosis, or leptospirosis.

26. The method according to claim 19, wherein said diseases or symptoms are: arrhythmia, rheumatic heart disease, angina pectoris, coronary atherosclerosis, hyperlipemia, hypercholesterolemia, hypertension, myocarditis, endocarditis, pericarditis, cardiac neurosis, cerebrovascular spasm, cerebral thrombosis, or peripheral vascular disease.

27. The method according to claim 19, wherein said diseases or symptoms are: aplastic anemia, nutritional anemia, hemolytic anemia, Mediterranean anemia, hemophilia, dysfunction of blood coagulation, thrombocytopenic purpura, granulocytopenia, leukemia, or leukopenia due to chemotherapy and radiotherapy.

28. The method A method according to claim 1, wherein said diseases and/or symptoms are: diabetes mellitus, uremia, nephritis, or nephrotic syndrome.

29. The method according to claim 19, wherein said diseases or symptoms are: anxiety, depression, Huntington's disease, Parkinson's disease, Alzheimer's disease (senile dementia) or presenile dementia.

30. The method according to claim 1, wherein said diseases or symptoms are: osteomyelitis, encephalitis, optic neuritis, conjunctivitis, otitis media, paranasal sinusitis, stomatitis, pharyngitis, gingivitis, periodontitis, tonsillitis, bronchitis, pneumonia, gastroenteritis, cholecystitis, cystitis, pancreatitis, arteritis, phlebitis, neuritis, vaginitis, vulvitis, orchitis, salpingitis, ovaritis, endometritis, prostatitis, or psoriasis.

31. The method according to claim 19, wherein said diseases or symptoms are: systemic lupus erythematosus, polymyositis, dermatomyositis, dermatasclerosis, rheumatoid arthritis, rheumatic heart disease, rheumatic arthritis, ulcerative disease, ulcerative colitis, pemphigus, allergic rhinitis, allergic dermatitis, allergic purpura, asthma, serum disease, or allergic granulocytopenia.

32. The method according to claim 19, wherein said diseases or symptoms are: viral dermatosis, coccigenic dermatosis, bacillary dermatosis, fungal dermatosis, spirochetal dermatosis, zoogenetic dermatosis, occupational dermatosis, neurotic dermatosis, allergodermia, physicogenic dermatosis, photogenous dermatosis, allergic dermatosis, lichen planus and lichenoid rash, erythemoid dermatosis, erythroderma desquamativum, vesicular dermatosis, keratinizing dermatosis, pigmentary dermatosis, dermovascular disease and lymphodermia, nutritional and metabolic dermatosis, disease of dermis and hypodermis, reticulosis, skin syndrom, granuloma annulare, pustuloderma, dermatitis continuee of extremities, edeomycodermatitis such as cervical erosion, neurodermatitis, or ulcerative disease of gastrointestinal tract and mouth cavity.

33. The method according to claim 19, wherein said diseases or symptoms are: drug addiction to papaverine, morphine, heroin, cocaine, “ice” (methamphetamine hydrochloride), opium, or dolantin.

34. The method according to claim 19, wherein said diseases or symptoms are: burn, scald, cold injury, trauma, bone injury, soft tissue injury, snakebite, or bee sting.
